(19) United States
(12) Patent Application Publication
(10) Pub. No.: US 2003/0199446 A1
(43) Pub. Date: Oct. 23, 2003

(54) Use of ectoin or ectoin derivatives for stabilizing p53

(76) Inventors: Joachim Bunger, Gross-Umstadt (DE); Francois Marchio, Scarsdale, NY (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/363,469

(22) PCT Filed: Aug. 21, 2001
(86) PCT No.: PCT/EP01/09670

(30) Foreign Application Priority Data
Sep. 4, 2000 (DE) 100 43 456.8

(51) Int. Cl. A61K 38/17; A61K 48/00

(52) U.S. Cl. 514/12; 514/44; 514/269

(57) ABSTRACT

This invention relates to the use of at least one compound, selected from a compound of the formula 1a, 1b,

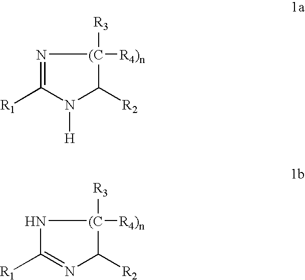

a physiologically compatible salt of it and a stereoisomeric form of it, whereby the following signify

R¹ H or alkyl,

R² H, COOH, COO-alkyl or CO—NH—R⁵,

R³ and R⁴ each mutually independent H or OH,

n 1, 2 or 3,

R⁵ H, alkyl, an amino acid residue, a dipeptide residue or tripeptide residue, and

alkyl an alkyl residue with 1 to 4 carbon atoms,

for the stabilisation of p53.

According to the invention, these compounds are normally used in the form of a topical composition.

The present invention relates to the use of ectoin or ectoin derivatives in the stabilisation of p53.

In western industrial countries cancer has developed into one of the most feared illnesses. This is in part due to the fact that still no effective therapy has been found for some types of cancer or that the relevant cancer has become resistant to either chemotherapy or radiotherapy. It is assumed that cancer is the cause of death of ⅕th of people in the western industrial countries.

The process of cancer formation is known as cancerogenesis. According to current thinking, the cells of a tumour form from a common stem cell (clonality). The process in which a cell degenerates to a cancer cell, i.e. a malignant transformation occurs, is attributed to the circumvention or a disturbance of the normal cell growth control. With defective cell divisions, rearrangement of chromosomes or parts of chromosomes can take place without restoration through internal cell repair mechanisms. This results in the activation of existing genes with primary importance for the regulation of cell activities, so-called oncogens. These oncogens lead to a disturbance in growth control, e.g. due to the production of growth factors which in turn stimulate the cell. The uncontrolled increase in cell proliferation gives rise to biochemical and physical changes, leading to the further loss of growth inhibition (autonomy), to cellular and histological abnormalities and to dedifferentiation (anaplasia) as well as spreading over the whole organism (metastases).

The conditions thought to cause the formation of cancer are genetic factors, ionising radiation, UV light, viruses and the influence of carcinogens in the form of tobacco smoke, nutrition, medication or due to ingestion at the work-place or in the environment. Also the lack of resistance to tumour cells as a consequence of disturbances in the immune system contributes to the formation of cancer illnesses. This is also the approach of psychophysiological theories which take into account the effect of stress and psychological factors.

The link between cancerogenesis and mutagenesis has been established for three cancer factors:

chemical carcinogens which cause simple local changes in the DNA sequence;

ionising radiation which can cause chromosome breakage and translocations; and

viruses which introduce external DNA into the cells.

It is assumed that the cell possesses mechanisms to prevent the external DNA of viruses and bacteria from being passed to its daughter cells. This has meant that cancer researchers have turned to tumour and growth suppression genes, in particular the p53 gene.

The p53 gene is located on the short arm of the human Chromosome 17, Band 13 and is approximately 20 kilobases (kb) long. This gene gives a 2.8 kb mRNA transcript and codes for a 53 kD phosphor protein which contains 393 amino acids.

The p53 protein is able to bind to special sequences and it is assumed that it is a transcription factor.

The p53 reacts to DNA damage or abnormal growth conditions in that it can stop the cell in the G1 phase of the DNA replication (interphase). The p53 protein synthesis is reinforced due to DNA strand breakage and AT gene products. The p53 proteins, for their part, reinforce the formation of negative growth factors and inhibit the DNA replication, positive growth factors and GTP synthesis via IMP dehydrogenase.

A further aspect of this type of regulation includes the control function of p53 in the cell, where if the cell becomes damaged too much, controlled cell death or apoptosis is induced before these cells can grow into tumours. Therefore, p53 has a certain role in the cell response to UV radiation due to the inhibition of the DNA synthesis, followed by DNA damage.

With the most frequently observed genetic change in human malignant tumour cells, changes to the p53 gene and its coded protein are involved. Especially, the genetic factors for p53 are particularly frequently mutated in skin tumour cells where these have been caused by UV light. Excessive solar radiation can cause the formation of skin tumours.

A cell deficient in p53 or which expresses mutated p53 does not enter the G1 arrest or G0 (apoptosis) phase. The incapability of the mutated p53 to initiate apoptosis may also explain why radiation therapy is ineffective in the treatment of various tumour cells.

There are various methods of how the p53 function can be deactivated. These include missense mutations, deletions or nonsense mutations of the gene which lead to the protein not being able to oligomerise or form tetramer complexes which can bind to special DNA sequences.

The p53 gene has a number of “hot spots”, i.e. gene sections which can quickly mutate. Furthermore, the p53 gene is very sensitive with respect to UV radiation which can easily lead to a mutation and the resulting loss of function of the p53.

Since the significance of p53 has been recognised, particularly in the formation of skin tumours, suggestions have already been made of how the watchman function of p53 can also operate in skin cells in which the p53 gene has mutated due to strong solar radiation. For example, Bild der Wissenschaft 2196, page 108, reports that it has been suggested that biotechnically produced p53 protein is packaged in a lipid envelope so that it can be transported to the skin cells. This p53 preparation in the form of a cream is intended to eradicate potential cancer cells and possibly even regress existing skin cancer. However, this procedure through the application of p53 itself is very complicated and expensive.

It is therefore the object of this invention to make available a compound which stabilises p53 on the DNA and protein level. It is thereby intended to reduce a mutation and a subsequent loss of the p53 function to be able to initiate apoptosis also with tumour cells. At the same time, the synthesis of p53 is to be stimulated, so that under stress conditions such as UV radiation and chemical noxae, p53 is available in sufficient concentration.

This object is resolved by the use of at least one compound, selected from a compound of the formula 1a, 1b,

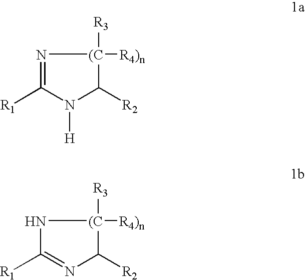

a physiologically compatible salt of it and a stereoisomeric form of it, whereby the following signify

R¹ H or alkyl,

R² H, COOH, COO-alkyl or CO—NH—R⁵,

R³ and R⁴ each mutually independent H or OH,

n 1, 2 or 3,

R⁵ H, alkyl, an amino acid residue, a dipeptide residue or tripeptide residue, and

alkyl an alkyl residue with 1 to 4 carbon atoms,

for the stabilisation of p53.

The compounds in the formulas 1a and 1b, the physiologically compatible salts of the compounds in the formulas 1a and 1b and the stereoisomeric form of the compounds in the formulas 1a and 1b are also termed in the following as “ectoin or ectoin derivatives”.

Ectoin and ectoin derivatives involve low molecular, cyclic amino acid derivatives which can be obtained from various halophile microorganisms. Both ectoin and ectoin derivatives have the advantage that they do not interfere with the cell metabolism. Ectoin and ectoin derivatives have already been described in DE 43 42 560 as moisturisers in cosmetic products.

The compounds, as used in the invention, can be present in the topical compositions as optical isomers, diastereomers, racemate, amphoteric ions, cations or as a mixture of these.

As compounds used according to the invention, those are preferred where R¹ is H or CH₃, R² is H or COOH, R³ and R⁴ signify mutually independent H or OH and n is 2. Of the compounds used according to the invention, (S)-1,4,5,6-tetrahydro-2-methyl-4-pyrimidine carboxylic acid (ectoin) and (S,S)-1,4,5,6-tetrahydro-5-hydroxy-2-methyl-4-pyrimidine carboxylic acid (hydroxyectoin) are particularly preferred.

The term “amino acids” is taken to mean the stereoisomeric forms, e.g. D and L-forms of the following compounds: alanine, β-alanine, arginine, asparagine, aspartic acid, cystine, glutamine, glutamic acid, glycine, histidine, isoleucine, leucine, lysine, methionine, phenylalanine, serine, threonine, tryptophan, tyrosine, valine, γ-amino butyrate, Nε-acetyllysine, Nδ-acetylomithine, Nγ-acetyl diaminobutyrate and Nα-acetyl diaminobutyrate. L-amino acids are preferred.

Amino acid residues are derived from the corresponding amino acids.

The residues of the following amino acids are preferred: alanine, p-alanine, asparagine, aspartic acid, glutamine, glutamic acid, glycine, serine, threonine, valine, γ-amino butyrate, Nε-acetyllysine, Nδ-acetylomithine, Nγ-acetyl diaminobutyrate and Nα-acetyl diaminobutyrate.

According to their chemical nature, the di- and tripeptide residues are acid amides and decompose during hydrolysis into two or three amino acids. The amino acids in the di- and tripeptide residues are linked together through amide bonds. Preferred di- and tripeptide residues are formed from the preferred amino acids.

The alkyl groups include the methyl group CH₃, the ethyl group C₂H₅, the propyl groups CH₂CH₂CH₃ and CH(CH₃)₂ as well as the butyl groups CH₂CH₂CH₂CH₃, H₃CCHCH₂CH₃, CH₂CH(CH₃)₂ and C(CH₃)₃. The preferred alkyl group is the methyl group.

Preferred physiologically compatible salts of the compounds used according to the invention are, for example, alkali, alkali earth or ammonia salts such as Na, K, Mg or Ca salts as well as salts which are derived from the organic bases triethylamine or tris(2-hydroxy-ethyl)amine. Further preferred physiologically compatible salts of the compounds used according to the invention are produced by reaction with inorganic acids such as hydrochloric acid, sulphuric acid and phosphoric acid or with organic carboxylic or sulfonic acids, such as acetic acid, citric acid, benzoic acid, maleic acid, fumaric acid, tartaric acid and p-toluenesulfonic acid.

Compounds in the formulas 1a and 1b, in which basic and acid groups, such as carboxyl or amino groups, are present in the same number, form the inner salts.

The manufacture of the compounds used according to the invention is described in DE 43 42 560. (S)-1,4,5,6-tetrahydro-2-methyl-4-pyrimidine carboxylic acid or (S,S)-1,4,5,6-tetrahydro-5-hydroxy-2-methyl-4-pyrimidine carboxylic acid can also be obtained microbiologically (Severin et al., J. Gen. Microb. 138 (1992) 1629-1638).

Ectoin or ectoin derivatives are normally, according to the invention, used in the form of a topical composition. It is also possible to use them in the pharmaceutical field and/or in the field of nutrition.

The manufacture of the topical composition occurs in that at least one of the compounds used according to the invention, is, where applicable, brought into a suitable formulation form with auxiliary and/or carrier products. The auxiliary and carrier products originate from the group of carrier materials, preservatives and other usual auxiliary products.

The topical composition on the basis of at least one compound used according to the invention is applied externally to the skin or the skin adnexae.

The following are mentioned as forms of application: Solutions, suspensions, emulsions, pastes, ointments, gels, creams, lotions, powders, soaps, oils, sprays and cleaning preparations containing tenside. In addition to one or more compounds used according to the invention, any usual carrier products, auxiliary products and, where applicable, further active ingredients, can be added to the composition.

Preferred auxiliary products originate from the group of preservatives, antioxidants, stabilisers, solubilisers, vitamins, colouring agents and deodorisers. Ointments, pastes, creams and gels can contain, along with one or more compounds used according to the invention, the usual carrier products, e.g. animal and vegetable fats, waxes, paraffins, starch, traganth, cellulose derivatives, polyethylene glycols, silicones, bentonites, silicic acid, talcum and zinc oxide or mixtures of these substances.

Powders and sprays can contain, apart from one or more compounds used according to the invention, the usual carrier products, e.g. lactose, talcum, silicic acid, aluminium hydroxide, calcium silicate, polyamide powder or mixtures of these substances. Sprays can additionally contain the usual propellants, e.g. chloro-fluoro hydrocarbons, propane/butane or dimethyl ether.

Solutions and emulsions can contain, apart from one or more compounds used according to the invention, the usual carrier products, such as solvents, solubilisers and emulsifiers, e.g. water, ethanol, isopropanol, ethyl carbonate, ethyl acetate, benzyl alcohol, benzyl benzoate, propylene glycol, 1,3-butyl glycol, oils, in particular cotton seed oils, peanut oil, corn oil, olive oil, castor oil, sesame oil, glycerol fatty acid ester, polyethylene glycols and sorbitan fatty acid ester or mixtures of these substances.

Suspensions can contain, apart from one or more compounds used according to the invention, the usual carrier products, such as liquid diluting agents, e.g. water, ethanol or propylene glycol, suspension agents, e.g. ethoxylated isostearyl alcohols, polyoxyethylene sorbitane ester, microcrystalline cellulose, aluminium metahydroxide, bentonite, agar-agar and traganth or mixtures of these substances.

Soaps can contain, apart from one or more compounds used according to the invention, the usual carrier products, such as alkali salts of fatty acids, salts of fatty acid half esters, fatty acid protein hydrolysates, isothionates, lanolin, fatty alcohol, plant oils, plant extracts, glycerine, sugar or mixtures of these substances.

Cleaning products containing tenside can contain, apart from one or more compounds used according to the invention, the usual carrier products, such as salts of fatty alcohol sulphates, fatty alcohol ether sulphates, sulfo succinic acid half esters, fatty acid protein hydrolysates, isothionates, imidazolinium derivatives, methyl taurates, sarcosinates, fatty acid amide ether sulphates, alkyl amidobetaine, fatty alcohols, fatty acid glycerides, fatty acid diethanol amides, plant and synthetic oils, lanolin derivatives, ethoxylated glycerine fatty acid esters or mixtures of these substances.

Face and body oils can contain, apart from one or more compounds used according to the invention, the usual carrier products, such as synthetic oils like fatty acid esters, fatty alcohols, silicone oils, natural oils like plant oils and oily plant extracts, paraffin oils, lanolin oils or mixtures of these substances.

Further typical cosmetic forms of application are also lip sticks, lip care sticks, mascara, eyeliners, eye-shadows, rouge, powder, emulsion and wax make-up as well as sun protection, pre-sun and after-sun preparations.

At least one compound used according to the invention is present in the topical composition in an amount of preferably 0.0001 to 50% wt., particularly preferred 0.001 to 10% wt. and especially preferred 0.1 to 1% wt., referred to the composition.

Apart from ectoin or the ectoin derivatives, preferentially, in addition, at least one antioxidation agent and/or UV filter is used.

Antioxidation agents known from the specialist literature can be used according to the invention, e.g. flavonoids, coumaranone, amino acids, (e.g. glycine, histidine, tyrosine, tryptophan) and their derivatives, imidazoles (e.g. urocanicic acid) and their derivatives, peptides, such as D,L-carnosine, D-carnosine, L-carnosine and their derivatives (e.g. anserine), carotinoids, carotenes (e.g. α-carotene, β-carotene, lycopene) and their derivatives, chlorogenic acid and its derivatives, lipoic acid and its derivatives (e.g. dihydrolipoic acid), aurothioglucose, propylthiouracil and other thiols (e.g. thioredoxin, glutathione, cysteine, cystine, cystamine, and their glycosyl, N-acetyl, methyl, ethyl, propyl, amyl, butyl and lauryl, palmitoyl, oleyl, γ-linoleyl, cholesteryl and glyceryl esters) as well as their salts, diaurylthiodipropionate, distearylthiodipropionate, thiodipropionic acid and their derivatives (esters, ethers, peptides, lipides, nucleotides, nucleosides and salts) as well as sulfoximine compounds (e.g. buthionine sulfoximines, homocysteine sulfoximine, buthionine sulfones, penta, hexa, heptathionine sulfoximine), also (metal-) chelators (e.g. α-hydroxy fatty acids, palmitic acid, phytic acid, lactoferrine), α-hydroxy acids (e.g. citric acid, lactic acid, malic acid), humic acid, bile acid, bile extracts, bilirubin, biliverdin, EDTA, EGTA and their derivatives, unsaturated fatty acids and their derivatives, vitamin C and derivatives (e.g. ascorbyl palmitate, magnesium ascorbyl phosphate, ascorbyl acetate) as well as coniferyl benzoate of benzoin gum, rutinic acid and its derivatives, α-glucosyl rutin, ferulic acid, furfurylidene glucitol, carnosine, butyl hydroxyl toluol (BHT), butyl hydroxy anisol, nordihydroguaiaretic acid, trihydroxy butyrophenone, uric acid and its derivatives, mannose and its derivatives, zinc and its derivatives (e.g. ZnO, ZnSO₄), selenium and its derivatives (e.g. selenium methionine), stilbenes and their derivatives (e.g. stilbene oxide, trans-stilbene oxide).

Mixtures of antioxidation agents are also suitable. Well-known and commercial mixtures are, for example, mixtures containing as active ingredient lecithin, L-(+)-ascorbyl palmitate and citric acid (e.g. Oxynex® AP), natural tocopherols, L-(+)-ascorbyl palmitate, L-(+)-ascorbic acid and citric acid (e.g. Oxynex®) K LIQUID), tocopherol extracts from natural sources, L-(+)-ascorbyl palmitate, L-(+)-ascorbic acid and citric acid (e.g. Oxynex® L LIQUID), DL-α-tocopherol, L-(+)-ascorbyl palmitate, citric acid and lecithin (e.g. Oxynex® LM) or butyl hydroxy toluol (BHT), L-(+)-ascorbyl palmitate and citric acid (e.g. Oxynex® 2004).

Butyl hydroxy toluol is used as an antioxidation agent in a preferred embodiment of the invention. In a further preferred embodiment one or more compounds, selected from flavonoids and/or coumaranones, are used as antioxidation agent.

The glycosides of flavanones, flavones, 3-hydroxy flavones (=flavanols), aurones, isoflavones and rotenoids (Römpp Chemie-Lexikon, Band 9, 1993; [Römpp Chemical Dictionary Vol. 9, 1993]) are grouped under flavanoids. Within the scope of this invention however, this is also taken to include the aglycones, i.e. the sugar-free constituents, and the derivatives of the flavonoids and aglycones. Within the scope of this invention, the coumaranones are also taken to include their derivatives.

Preferred flavonoids are derived from flavanones, flavones, 3-hydroxy flavones, aurones and isoflavones, in particular from flavanones, flavones, 3-hydroxy flavones and aurones.

The flavanones are characterised by the following basic structure:

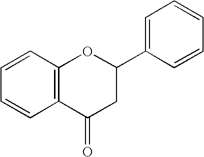

The flavones are characterised by the following basic structure:

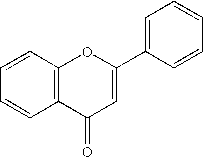

The 3-hydroxy flavones (flavonols) are characterised by the following basic structure:

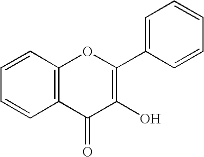

The isoflavones are characterised by the following basic structure:

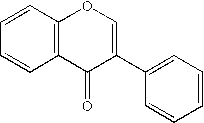

The aurones are characterised by the following basic structure:

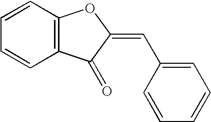

The coumaranones are characterised by the following basic structure:

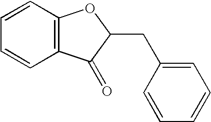

Preferentially, the flavonoids and coumaranones are selected from the compounds in formula (1):

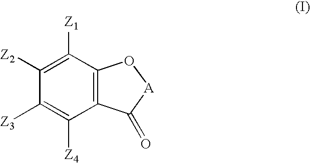

whereby the following signify:

Z₁ to Z₄ each indicate, independently of one another, H, OH, alkoxy, hydroxy alkoxy, mono or oligoglycoside residues, whereby the alkoxy and hydroxy alkoxy groups can be branched and unbranched and can exhibit 1 to 18 C atoms and whereby also sulphate or phosphate can be bonded to the hydroxy groups of the quoted residues,

A is selected from the group consisting of the subforms (IA), (IB) and (IC),

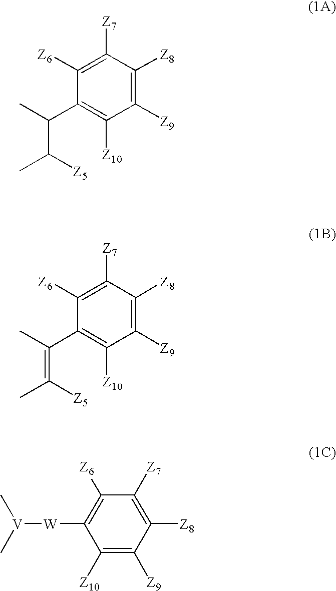

Z₅ H, OH or OR,

R a mono or oligoglycoside residue,

Z₆ to Z₁₀ possess the meanings of the residues Z₁ to Z₄, and

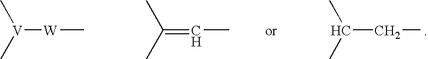

The alkoxy groups are preferably linear and possess 1 to 12, preferably 1 to 8 C atoms. These groups therefore correspond to the formula —O—(CH₂)ₘ—H where m signifies 1, 2, 3, 4, 5, 6, 7 or 8 and especially 1 to 5.

The hydroxy alkoxy groups are preferably linear and possess 2 to 12, preferably 2 to 8 C atoms. These groups therefore correspond to the formula —O—(CH₂)ₙ—OH where n signifies 2, 3, 4, 5, 6, 7 or 8, in particular 2 to 5 with 2 especially preferred.

The mono and oligoglycoside residues are preferably formed from 1 to 3 glycoside units. Preferably, these units are selected from the group of hexosyl residues, in particular the rhamnosyl and glucosyl residues. But also other hexosyl residues, for example, allosyl, altrosyl, galactosyl, gulosyl, idosyl, mannosyl and talosyl can be applied to advantage where applicable. According to the invention, it may also be advantageous to use pentosyl residues.

In a preferred embodiment the following meanings apply:

Z₁ and Z₃ signify H,

Z₂ and Z₄ have a meaning different to H, in particular they signify OH, methoxy, ethoxy or 2-hydroxy ethoxy,

Z₅ signifies H, OH or a glycoside residue formed from 1 to 3, but preferably 1 or 2 glycoside units,

Z₆, Z₉ and Z₁₀ signify H, and

Z₇ and Z₈ signify a meaning different to H, in particular they signify OH, methoxy, ethoxy or 2-hydroxy ethoxy.

In another preferred embodiment, in particular, when the water-solubility of the flavonoids and coumaranones is to be increased, a sulphate or phosphate group is bonded to the hydroxy groups. Suitable counterions are, for example, the ions of alkali or alkaline earth metals, whereby they are, for example, selected from sodium or potassium.

In another preferred embodiment the flavonoids are selected from the following compounds: 4,6,3′,4′-tetrahydroxy aurone, quercetin, rutin, isoquercetin, anthocyanidin (cyanidin), eriodictyol, taxifolin, luteolin, tris-hydroxy ethyl quercetin (troxequercetin), tris-hydroxy ethyl rutin (troxerutin), tris-hydroxy ethyl isoquercetin (troxe-isoquercetin), tris-hydroxy ethyl luteolin (troxeluteolin) and their sulphates and phosphates.

Amongst the flavonoids, rutin and troxerutin are particularly preferred. Troxerutin is especially preferred.

Amongst the coumaranones 4,6,3′,4′-tetrahydroxybenzyl coumaranone-3 is preferred.

The antioxidation agents are, according to the invention, used in usual amounts in the topical composition.

Furthermore, according to the invention, well-known UV filters from the specialist literature can be used.

As suitable organic UV filters, all UVA as well as UVB filters known to the specialist can be considered. There are many well-known and proven substances from the specialist literature for both UV ranges, e.g.:

benzylidene camphor derivatives, such as

3-(4′-methylbenzylidene)-di-camphor (e.g. Eusoiex® 6300),

3-benzylidene camphor (e.g. Mexoryl® SD),

polymers of N-{(2 and 4)-[(2-oxoborn-3-yliden)methyl]benzyl}acrylamide (e.g. Mexoryl®SW),

N,N,N-trimethyl-4-(2-oxoborn-3-ylidenmethyl)anilium-methyl sulphate (e.g. Mexoryl® SK) or

α-(2-oxoborn-3-yliden)toluol-4-sulphonic acid (e.g.Mexoryl® SL);

benzoyl or dibenzoylmethanes such as

1-(4-tert-butylphenyl)-3-(4-methoxyphenyl)propane-1,3-dion (e.g. Eusolex® 9020) or

4-isopropyl dibenzoylmethane (e.g. Eusolex® 8020);

benzophenones such as

2-hydroxy-4-methoxybenzophenone (e.g. Eusolex® 4360) or

2-hydroxy-4-methoxybenzophenone-5-sulphonic acid and its sodium salt (e.g. Uvinul® MS-40);

methoxy cinnamic acid esters such as

p-methoxy-cinnamic acid-2-ethylhexyl ester (e.g. Eusolex® 2292),

p-methoxy-cinnamic acid isopentyl ester, e.g. as a mixture of isomers (e.g. Neo Heliopan® E 1000);

salicylate derivatives, such as

2-ethylhexyl salicylate (e.g. Eusolex® OS),

4-isopropylbenzyl salicylate (e.g. Megasol®), or

3,3,5-trimethyl cyclohexyl salicylate (e.g. Eusolex® HMS);

4-amino-benzoic acid and its derivatives such as

4-amino-benzoic acid,

4-(dimethylamino)benzoic acid-2-ethylhexyl ester (e.g. Eusolex® 6007),

ethoxylated 4-amino-benzoic acid ethyl ester (e.g. Uvinul® P25);

and other substances such as

2-cyano-3,3-diphenylacrylic acid-2-ethylhexyl ester (e.g. Eusolex® OCR),

2-phenylbenzimidazol-5-sulfonic acid and its potassium, sodium and triethanol-amine salts (e.g. Eusolex®) 232),

3,3′-(1,4-phenylendimethylene)-bis-(7,7-dimethyl-2-oxobicyclo[2.2.1]hept-1-ylmethane sulfonic acid and its salts (e.g. Mexoryl® SX), and

2,4,6-trianilino-(p-carbo-2′-ethylhexyl-1′-oxi)-1,3,5-triazine (e.g. Uvinul® T 150).

These organic UV filters are normally used in an amount of 0.5 to 10% wt., preferably 1 to 8% wt. in the topical composition used according to the invention.

Further suitable organic UV filters are for example:

2-(2H-benzotriazol-2-yl)-4-methyl-6-(2-methyl-3-(1,3,3,3-tetramethyl-1-(trimethylsilyloxy)disiloxanyl)propyl)phenol (e.g. Silatrizole®),

4,4′-[(6-[4-((1,1-dimethylethyl)aminocarbonyl)phenylamino]-1,3,5-triazine-2,4-diyl)diimino]bis(benzoic acid-2-ethylhexyl ester) (e.g. Uvasorb® HEB),

α-(trimethylsilyl)-ω[trimethylsiyl)oxy]poly[oxy(dimethyl] [and approx. 6% of methyl[2-[p-[2,2-bis(ethoxycarbonyl]vinyl]phenoxy]-1-methylene ethyl] and approx. 1.5% of methyl[3-[p-[2,2-bis(ethoxycarbonyl)vinyl)phenoxy)-propenyl) and 0.1 to 0.4% of (methylhydrogen]silylene]] (n≈60) (e.g. Parsol® SLX),

2,2′-methylene-bis-(6-(2H-benzotriazol-2-yl)-4-(1,1,33-tetramethylbutyl)phenol (e.g. Tinosorb® M),

2,2′-(1,4-phenylene)bis-(1H-benzimidazol-4,6-disulphonic acid, monosodium salt,

2,2′-(1,4-phenylene)bis-(1H-benzimidazol-5-sulphonic acid, monosodium salt,

2,2′-(1,4-phenylene)bis-(1H-benzimidazol-5-sulphonic acid,

monopotassium salt, and

2,4-bis{[4-(2-ethyl-hexyloxy)-2-hydroxyl]-phenyl}-6-(4-methoxyphenyl)-1,3,5-triazine (e.g. Tinosorb® S).

These organic filters are normally used in an amount from 0.5 to 20% wt., preferably 1 to 15% wt., in the topical composition used according to the invention.

As inorganic UV filters those from the group of titanium dioxides, e.g. coated titanium dioxide (e.g. Eusolex® T-2000 or Eusolex® T-Aqua), zinc oxides (e.g. Sachtotec®), iron oxides or also ceric oxides can be considered. These inorganic UV filters are normally used in an amount from 0.5 to 20% wt., preferably 2 to 10% wt., in the topical composition used according to the invention.

Preferred UV filters are zinc oxide, titanium dioxide, 3-(4′-methylbenzylidene)-di-camphor, 1-(4-tert-butylphenyl)-3-(4-methoxyphenyl)propane-1,3-dion, 4-isopropyldibenzoylmethane, 2-hydroxy-4-methoxybenzophenone, methoxy cinnamic acid octyl ester, 3,3,5-trimethylcyclohexyl salicylate, 4-(dimethylamino)benzoic acid-2-ethylhexyl ester, 2-cyano-3,3-diphenylacrylic acid-2-ethylhexyl ester, 2-phenylbenzimidazol-5-sulfonic acid and its potassium, sodium and triethanolamine salts.

Especially preferred UV filters are zinc oxide and titanium dioxide.

If titanium dioxide is used according to the invention, it is preferable that, apart from titanium dioxide, additionally one or more other UV filters are used, selected from 3-(4′-methylbenzylidene)-di-camphor, 1-(4-tert-butylphenyl)-3-(4-methoxyphenyl)propane-1, 3-dion, 4-isopropyldibenzoyl-methane, 2-hydroxy-4-methoxybenzophenone, methoxy cinnamic acid octyl ester, 3,3,5-trimethylcyclohexyl salicylate, 4-(dimethylamino)benzoic acid-2-ethylhexyl ester, 2-cyano-3,3-diphenylacrylic acid-2-ethylhexyl ester, 2-phenylbenzimidazol-5-sulfonic acid and its potassium, sodium and triethanolamine salts.

It is especially preferred that, apart from titanium dioxide, the UV filters 2-hydroxy-4-methoxybenzophenone and/or p-methoxy cinnamic acid-2-ethylhexyl esters are additionally used.

Ectoin and ectoin derivatives can, according to the invention, be used as medicaments for stabilising p53. A prophylactic application, i.e. an application before a stress loading such as UV light or chemical noxae or a therapeutic application following this stress loading, e.g. as an “after-sun preparation”, can be considered. A cosmetic application and an application in the nutritional field are also possible. The use of ectoin or ectoin derivatives according to the invention here leads to a stabilisation of p53 at the DNA and protein levels in the cells so that the natural repair and protection mechanisms of the skin and other tissue are improved. The mutation of the p53 gene due to UV light or chemical noxae can be extensively prevented by ectoin or ectoin derivatives, so that cells damaged by cancer can be killed off by p53 before they grow to form a cancerous focus. Furthermore, ectoin and ectoin derivatives lead to a higher concentration of p53 under stress conditions, because ectoin stimulates the synthesis of p53. The p53 protein is protected by ectoin or ectoin derivatives in that the active substance forms a hydrate coat around the protein. This leads to the water molecules not being able to be removed from the protein structure of p53, so that the 3D structure of the p53 protein is preserved. Consequently, overall an improvement of the defensive status of the cells arises, particularly with the skin cells.

The following formulation examples explain this invention. All compounds or components which can be used in the cosmetic formulations are either known and commercially available or can be synthesized according to known methods.

The INCI names of the raw materials used are as follows (the INCI names are given in English according to the definition):

| Raw Material | INCI Name |
|---|---|
| Almond oil | Sweet almond oil (prunus dulcis) |
| Eutanol G | Octyldodecanol |
| Luvitol EHO | Cetearyl octanoate |
| Oxynex K liquid | PEG-8, tocopherol, ascorbyl palmitate, |
| | ascorbic acid, citric acid |
| Panthenol | Panthenol |
| Karion F liquid | Sorbitol |
| Sepigel 305 | Polyacrylamide, C13-14 isoparaffin, |
| | laureth-7 |
| Paraffin, low viscosity | Mineral oil (paraffinum liquidum) |
| Mirasil CM 5 | Cyclomethicone |
| Arlacel 165 | Glyceryl stearate, PEG-100 stearate |
| Germaben II | Propylene glycol, diazolidinyl urea, |
| | methylparaben, propylparaben |
| Perfume Bianca | Parfum |
| Abil WE 09 | Polyglyceryl-4 isostearate, cetyl |
| | dimethicone copolyol, hexyl laurate |
| Jojoba oil | Jojoba oil (buxus chinensis) |
| Cetiol V | Decyl oleate |
| Prisorine IPIS 2021 | Isopropyl isostearate |
| Castor oil | Castor oil (ricinus communis) |
| Lunacera M | Cera microcristallina |
| Miglyol 812 neutral oil | Caprylic/capric triglyceride |
| Eusolex T-2000 | Titanium dioxide, alumina, simethicone |

### EXAMPLE 1

A skin-care gel (O/W), containing ectoin, is made from the following components:

| | | | % wt. |
|---|---|---|---|
| A) | Almond Oil | (2) | 8.0 |
| | Eutanol G | (3) | 2.0 |
| | Luvitol EHO | (4) | 6.0 |
| | Oxynex K liquid (Art. No. 108324) | (1) | 0.05 |
| B) | Panthenol (Art. No. 501375) | (1) | 0.5 |
| | Karion F liquid (Art. No. 102993) | (1) | 4.0 |
| | Preservative | | q.s. |
| | Water, demineralised | | ad 100 |
| C) | Sepigel 305 | (5) | 3.0 |
| D) | RonaCare ™ Ectoin | (1) | 1.0 |

The following can be used as preservative:

0.05% propyl-4-hydroxybenzoate (Art. No. 107427) or

0.15% methyl-4-hydroxybenzoate (Art. No. 106757)

Manufacture:

The combined Phase B is slowly introduced into Phase C under slow stirring. Then the predissolved Phase A is added. It is stirred until the phases are homogeneously mixed. Then Phase D is added and it is stirred until homogeneity is obtained.

Procurement Sources:

(1) Merck KGaA, Darmstadt

(2) Gustav Heess, Stuttgart

(3) Henkel KGaA, Düsseldorf

(4) BASF AG, Ludwigshafen

(5) Seppic, France

### EXAMPLE 2

A skin-care cream (O/W), containing ectoin, is made from the following components:

| | | | % wt. |
|---|---|---|---|
| A) | Paraffin, low viscosity (Art. No. 107174) | (1) | 8.0 |
| | Isopropylmyristate (Art. No. 822102) | (1) | 4.0 |
| | Mirasil CM 5 | (2) | 3.0 |
| | Stearic acid | (1) | 3.0 |
| | Ariacel 165 | (3) | 5.0 |
| B) | Glycerin, 87% (Art. No. 104091) | (1) | 3.0 |
| | Germaben II | (4) | 0.5 |
| | Water, demineralised | | ad 100 |
| C) | Perfume Bianca | (5) | 0.3 |
| D) | RonaCare ™ Ectoin | (1) | 11.0 |

Manufacture:

First, the Phases A and B are heated separately to 75° C. Then Phase A is slowly added to Phase B under stirring and stirred until a homogeneous mixture is obtained. After the emulsion has been homogenised, it is cooled under stirring to 30° C., the Phases C and D are added and stirring occurs until homogeneity is obtained.

Procurement Sources:

(1) Merck KGaA, Darmstadt

(2) Rhodia

(3) ICI

(4) ISP

(5) Dragoco

### EXAMPLE 3

A sun-protecting lotion (W/O), containing ectoin, is made from the following components:

| | | | % wt. |
|---|---|---|---|
| A) | Abil WE 09 | (2) | 5.0 |
| | Jojoba oil | (3) | 6.0 |
| | Cetiol V | (4) | 6.0 |
| | Prisorine 2021 | (5) | 4.5 |
| | Castor oil | (6) | 1.0 |
| | Lunacera M | (7) | 1.8 |
| | Miglyol 812 neutral oil | (8) | 4.5 |
| B) | Eusolex T-2000 (Art. No. 105373) | (1) | 3.0 |
| | Glycerin, 87% (Art. No. 104091) | (1) | 2.0 |
| | Sodium chloride (Art. No. 106400) | (1) | 0.4 |
| | Preservative | | q.s. |
| | Water, demineralised | | ad 100 |
| C) | Perfume | (5) | 0.3 |
| D) | RonaCare ™ Ectoin | (1) | 1.0 |

The following can be used as preservative:

0.05% propyl-4-hydroxybenzoate (Art. No. 107427) or

0.15% methyl-4-hydroxybenzoate (Art. No. 106757)

Manufacture:

First, Eusolex T-2000 is introduced into Phase B under stirring and heated to 80° C. Then Phase A is heated to 75° C. and Phase B is slowly added under stirring. Stirring occurs until homogeneity is obtained and then cooling under stirring to 30° C. Thereafter, the Phases C and D are added and stirring occurs until homogeneity is obtained.

Procurement Sources:

(1) Merck KGaA, Darmstadt

(2) Th. Goldschmidt AG, Essen

(3) H. Lamotte, Bremen

(4) Henkel, KGaA, Dusseldorf

(5) Unichema, Emmerich

(6) Gustav Heess, Stuttgart

(7) H. B. Füller, Lüneburg

(8) Hüls Troisdorf AG, Witten

### EXAMPLE 4

A skin-care cream (O/W), containing ectoin, is made from the following components:

| | | | % wt. |
|---|---|---|---|
| A) | Paraffin, low viscosity (Art. No. 107174) | (1) | 8.0 |
| | Isopropylmyristate (Art. No. 822102) | (1) | 4.0 |
| | Mirasil CM 5 | (2) | 3.0 |
| | Stearic acid | (1) | 3.0 |
| | Arlacel 165 V | (3) | 5.0 |
| B) | Glycerin, 87% (Art. No. 104091) | (1) | 3.0 |
| | Germaben II | (4) | 0.5 |
| | Water, demineralised | | ad 100 |
| C) | RonaCare ™ Ectoin | (1) | 2.5 |

Manufacture:

First, the Phases A and B are heated separately to 75° C. Then Phase A is slowly added to Phase B under stirring and stirred until a homogenous mixture is obtained. After homogenisation of the emulsion, cooling to 30° C. occurs under stirring, Phase D is added and stirring takes place until homogeneity is obtained.

Procurement Sources:

(1) Merck KGaA, Darmstadt

(2) Rhodia

(3) ICI

(4) ISP

### EXAMPLE 5

Hair Tonic with Ectoin

| RAW MATERIAL | | INCI | % wt. |
|---|---|---|---|
| MERCARE ® Biotin | (1) | Biotin | 0.05 |
| Art. No. 130220 | | | |
| RonaCare ™ Ectoin | | (Ectoin) | 1.00 |
| Art. No. 130200 | | | |
| Octopirox | (2) | Piroctone Olamine | 0.10 |
| D(+)pantothenyl alcohol | (3) | Panthenol | 0.30 |
| (Art. No. 501375) | | | |
| Salicylic acid | (1) | Salicylic acid | 0.10 |
| (Art. No. 100631) | | | |
| N-cetyl-N,N,N-trimethyl-ammonium- | (1) | Cetrimonium bromide | 0.10 |
| bromide | | | |
| (Art. No. 102343) | | | |
| Dragoplant Hamamelis | (4) | Aqua, alcohol dentat., hamamelis | 1.00 |
| | | virginiana | |
| 2-propanol | (1) | Isopropyl alcohol | 45.00 |
| (Art. No. 100995) | | | |
| Demin. water | | Aqua | ad 100 |

Manufacture:

Biotin was dissolved in water and 2-propanol. Then ectoin was dissolved and the remaining raw materials added under stirring.

Procurement Sources:

(1) Merck KGaA

(2) Hoechst

(3) BASF

(4) Dragoco

### EXAMPLE 6

2 in 1 Shampoo

| RAW MATERIAL | | INCI | % wt. |
|---|---|---|---|
| Jaguar C-162 | (2) | Hydroxypropyl guar | 0.20 |
| | | Hydroxypropyltrimonium chloride | |
| Miranol Ultra C32 | (2) | Sodium cocoamphoacetate | 10.00 |
| Texapon NSO | (3) | Sodium laureth sulphate | 32.00 |
| Nicotinamide (vitamin B3) | (1) | Niacinamide | 0.10 |
| (Art. No. 130179) | | | |
| (D+)-biotin (vitamin H) | (1) | Biotin | 0.05 |
| (Art. No. 130220) | | | |
| RonaCare ™ Ectoin | (1) | (Ectoin) | 1.00 |
| (Art. No 130200) | | | |
| D-panthenol | (4) | Panthenol | 0.50 |
| Sodium chloride | (1) | Sodium chloride | 1.0 |
| (Art. No. 106400) | | | |
| Perfume | | Parfum | |
| Preservative | | | q.s. |
| Citric acid | (1) | Citric acid | q.s. |
| (Art. No. 130137) | | | |
| Demin. water | | Aqua | ad 100 |

Manufacture:

Jaguar C-162 was dispersed in water and hydrated with citric acid. The remaining raw materials were added under stirring in the stated sequence. Then the viscosity was adjusted with NaCl and the pH value with citric acid.

Procurement Sources:

(1) Merck KGaA

(2) Rhodia

(3) Cognis GmbH

(4) BASF AG

### EXAMPLE 7

Hair Styling Gel

| RAW MATERIAL | Art. No. | | INCI | % wt. |
|---|---|---|---|---|
| A | | | | |
| Pearl gloss pigments | | (1) | | 1.00 |
| Carbopol ETD 2001 | | (2) | Carbomer | 0.50 |
| 2-propanol for adj. | 1.09634 | (1) | Isopropyl alcohol | 20.00 |
| Water, demineralised | | | Aqua (water) | 30.00 |
| B | | | | |
| Luviskol K 30 Powder | | (3) | PVP | 1.60 |
| Germaben II | | (4) | Propylene glycol, diazolidinyl | 0.20 |
| | | | urea, methyl paraben, | |
| | | | propylparaben | |
| Triethanolamine, high | 108377 | (1) | Triethanolamine | 1.20 |
| purity | | | | |
| RonaCare ™ Ectoin | 130200 | (1) | (Ectoin) | 1.00 |
| Water, demineralised | | | Aqua (water) | 45.60 |

Manufacture:

The pearl gloss pigment was dispersed in the water/propanol mixture of Phase A and the Carbopol was sprinkled in under stirring. After complete dissolving, the predissolved Phase B was stirred in slowly.

Remarks:

Recommended pearl gloss pigments are interference pigments, silver pigments, gold pigments, iron oxide pigments.

Procurement Sources:

(1) Merck KGaA

(2) BF Goodrich GmbH

(3) BASF AG

(4) ISP Global Technologies

### EXAMPLE 8

Syndet Washing Tablet

| RAW MATERIAL | | INCI | % wt. |
|---|---|---|---|
| Zetasap 813A | (2) | Disodium lauryl sulfosuccinate, | 90.0 |
| | | sodium cocoyl isothionate, | |
| | | cetearyl alcohol, corn starch, | |
| | | glyceryl stearate, paraffin, | |
| | | titanium dioxide | |
| RonaCare ™ Ectoin | (1) | (Ectoin) | 1.00 |
| (Art. No. 130200) | | | |
| Perfume | | Parfum | 1.00 |
| Demin. water | | Aqua (water) | 8.00 |

Procurement Sources:

(1) Merck KGaA

(2) Zschimmer & Schwarz

### EXAMPLE 9

Shower Gel

| RAW MATERIAL | Art. No. | | INCI | % wt. |
|---|---|---|---|---|
| A | | | | |
| Timiron Splendid Green | 1.17477 | (1) | Cl 77891 (titanium dioxide), | 0.10 |
| | | | mica, silica | |
| Keltrol T | | (2) | Xanthan gum | 0.75 |
| Water, demineralised | | | Aqua (water) | 62.10 |
| B | | | | |
| Plantacare 2000 | | (3) | Decyl glucoside | 20.00 |
| Texapon ASV | | (3) | Magnesium oleth sulphate, | 0.65 |
| | | | sodium oleth sulphate, | |
| | | | magnesium laureth-8 | |
| | | | sulphate, sodium laureth-8 | |
| | | | sulphate, magnesium laureth | |
| | | | sulphate, sodium laureth | |
| | | | sulphate | |
| Bronidox L | | (3) | Propylene glycol 5-bromo-5- | 0.20 |
| | | | nitro-1,3-dioxane | |
| Perfume oil Everest | | (4) | Parfum | 0.05 |
| 79658 SB | | | | |
| RonaCare ™ Ectoin | 130200 | (1) | (Ectoin) | 1,00 |
| C | | | | |
| Citric acid monohydrate | 130137 | (1) | Citric acid | 0.15 |
| Water, demineralised | | | Aqua (water) | 10.00 |

Manufacture:

For Phase A the pigment was introduced into the water under stirring. Ketrol T was sprinkled in slowly under stirring and it was stirred until it was dissolved. The Phases B and C were added one after the other and slow stirring took place until everything was homogeneously distributed.

Procurement Sources:

(1) Merck KGaA

(2) Kelco

(3) Cognis GmbH

(4) Haanmann & Reimer GmbH

### EXAMPLE 10

Baby Powder

| RAW MATERIAL | Art. No. | | INCI | % wt. |
|---|---|---|---|---|
| A | | | | |
| IR 3535 TM | 111887 | (1) | Ethytbutylacetylaminopropionate | 4.00 |
| B | | | | |
| Magnesium hydroxide | 105827 | (1) | Magnesium carbonate | 10.00 |
| carbonate | | | hydroxide | |
| Dry Flo PC | | (2) | Aluminium starch | 86.00 |
| | | | Octenylsuccinate | |
| RonaCare ™ Ectoin | 130200 | (1) | (Ectoin) | 1.00 |

Manufacture:

Phase B was prepared and mixed with a propeller stirrer. Phase A was added drop by drop while stirring.

Procurement Sources:

(1) Merck KGaA

(2) National Starch & Chemical

### EXAMPLE 11

O/W After-Sun Lotion

| RAW MATERIAL | Art. No. | | INCI | % wt. |
|---|---|---|---|---|
| A | | | | |
| MERCARE ® Bisabolol | 130170 | (1) | Bisabolol | 0.30 |
| Montanov 68 | | (2) | Cetearyl alcohol | 4.00 |
| | | | Cetearyl glucoside | |
| Miglyol 812, neutral oil | | (3) | Caprylic/capric triglyceride | 12.00 |
| Mirasil CM5 | | (4) | Cyclomethicone | 2.00 |
| Mirasil DM 350 | | (4) | Dimethicone | 1.00 |
| B | | | | |
| Water, demineralised | | | Aqua (water) | 77.20 |
| Glycerin (87% high | 104091 | (1) | Glycerin | 3.00 |
| purity) | | | | |
| Preservative | | | | q.s. |
| RonaCare ™ Ectoin | 130200 | (1) | (Ectoin) | 1.00 |
| C | | | | |
| Rhodicare-S | | (4) | Xanthan gum | 0.50 |

Manufacture:

Phases A and B were heated separately to 75° C. Phase C was added slowly to B under stirring at 75° C. and stirring continued until a homogeneous mixture was obtained. Then Phase A was added to the B/C mixture and homogenised. The obtained mixture was cooled to room temperature under stirring.

Procurement Sources:

(1) Merck KGaA

(2) Seppic

(3) Hüls AG

(4) Rhodia GmbH

### EXAMPLE 12

Sun Protection Lotion (W/O)

| RAW MATERIAL | Art. No. | | INCI | % wt. |
|---|---|---|---|---|
| A | | | | |
| Eusolex 8300 | 105385 | (1) | 4-methylbenzylidene | 4.00 |
| | | | Camphor | |
| Eusolex 2292 | 105382 | (1) | Octylmethoxycinnamate, | 7.00 |
| | | | BHT | |
| Abil WE 09 | | (2) | Polyglyceryl-4-isostearate, | 5.00 |
| | | | Cetyl dimethicone copolyol, | |
| | | | Hexyl laurate | |
| Jojoba oil | | (3) | Buxus chinensis (jojoba oil) | 3.00 |
| Cetiol V | | (4) | Decyloleate | 3.00 |
| Prisorine 2021 | | (5) | Isopropyl isostearate | 2.00 |
| Paracera M | | (6) | Microwax | 1.00 |
| Miglyol 812, neutral oil | | (7) | Caprylic/capric triglyceride | 3.00 |
| Propyl-4- | 1.07427 | (1) | Propylparaben | 0.05 |
| hydroxybenzoate | | | | |
| B | | | | |
| Eusolex T-Aqua | 105401 | (1) | Aqua (water), | 16.00 |
| | | | titanium dioxide, alumina, | |
| | | | sodium metaphosphate, | |
| | | | phenoxyethanol, sodium | |
| | | | methylparaben | |
| Glycerin (87%, high | 104091 | (1) | Glycerin | 2.00 |
| purity) | | | | |
| Sodium chloride | 106400 | (1) | Sodium chloride | 0.40 |
| RonaCare ™ Ectoin | 130200 | (1) | (Ectoin) | 1.00 |
| Water, demineralised | | | Aqua (water) | 53.40 |
| Methyl-4- | 106757 | (1) | Methylparaben | 0.15 |
| hydroxybenzoate | | | | |

Manufacture:

Phase B was heated to 80° C. and Phase A to 75° C. Phase B was slowly introduced into Phase A under stirring. The mixture was homogenised and cooled under stirring.

Procurement Sources:

(1) Merck KGaA

(2) Th. Goldschmidt AG

(3) Henry Lamotte GmbH

(4) Cognis GmbH

(5) Unichema Chemie GmbH

(6) Paramelt

(7) Hüls AG

### EXAMPLE 13

Tooth Gel

| RAW MATERIAL | Art. No. | | INCI | % wt. |
|---|---|---|---|---|
| A | | | | |
| Sodium fluoride | 106441 | (1) | Sodium fluoride | 0.06 |
| Karion F liquid | 152698 | (1) | Sorbitol | 48.39 |
| Sodium benzoate | 106290 | (1) | Sodium benzoate | 0.16 |
| Sodium saccharinate | | | | 0.16 |
| RonaCare ™ Ectoin | 130200 | (1) | (Ectoin) | 1.00 |
| Water, demineralised | | | Aqua (water) | 29.12 |
| B | | | | |
| MERCARE ® Olaflur | 111680 | (1) | Olaflur, propylene glycol | 1.17 |
| Bromochlorophene | 1.03281 | (1) | Bromochlorophene | 0.08 |
| Aroma 35049 | | (2) | | 0.78 |
| C | | | | |
| Polyethylenglycol 400 | 807485 | (1) | PEG-8 | 2.34 |
| Tego Betain ZF | | (3) | Cocamidopropyl betaine | 3.89 |
| Sicomet Patent Blau | | (4) | | 0.62 |
| (E131), 0.1% in water | | | | |
| D | | | | |
| Sident 12 | | (5) | Silica | 7.40 |
| Sipemat 22 S | | (5) | Hydrated silica | 5.84 |

Manufacture:

Phases A and B were premixed separately. Phase C was heated to 50° C. Phases A and B were introduced into Phase C under stirring and mixed under vacuum. After slowly adding Phase D homogenisation took place under vacuum. Stirring continued under vacuum until the gel was clear.

Procurement Sources:

(1) Merck KGaA

(2) Crissa Drebing GmbH

(3) Th. Goldschmidt AG

(4) BASF AG

(5) Degussa AG

### EXAMPLE 14

Mouthwash Concentrate

| RAW MATERIAL | | % wt. |
|---|---|---|
| RonaCare ™ Ectoin | (1) | 1.00 |
| N-Cetylpyridiniumchloride (Art. No. 102340) | (1) | 0.50 |
| Ethanol (96%) (Art. No. 100971) | (1) | 70.00 |
| Peppermint aroma 77526-34 | (2) | 0.15 |
| Water, demineralised | | Ad 100.00 |

Manufacture:

All constituents were stirred until a clear solution was obtained.

Procurement Sources:

(1) Merck KGaA

(2) Givaudan-Roure, Dortmund

### EXAMPLE 15

Lip Salve

| RAW MATERIAL | | INCI | % wt. |
|---|---|---|---|
| RonaCare ™ Ectoin | (1) | (Ectoin) | 1.00 |
| (Art. No. 130200) | | | |
| Tagat S2 | (2) | PEG-20 glyceryl stearate | 10.00 |
| Lanette O | (3) | Cetearyl alcohol | 20.00 |
| Glycerin (87%) | (1) | Glycerin | 20.00 |
| (Art. Nr. 104091) | | | |
| Vaseline | (4) | Petrolatum | 35.00 |

Manufacture

All constituents were heated to 75° C. and then cooled to room temperature under stirring.

Procurement Sources:

(1) Merck KGaA

(2) Goldschmidt GmbH

(3) Cognis GmbH

(4) Schumann Sasol

### EXAMPLE 16

Lip Gloss

| RAW MATERIAL | Art. No. | | INCI | % wt. |
|---|---|---|---|---|
| A | | | | |
| Pearl gloss pigments | | (1) | | 10.00 |
| B | | | | |
| Indopol H 100 | | (2) | Polybutene | 59.95 |
| Bentone Gel MIO V | | (3) | Quatemium-18 hectorite, | 20.00 |
| | | | propylene carbonate, | |
| | | | paraffinum liquidum | |
| | | | (mineral oil) | |
| Eutanol G | | (4) | Octyldodecanol | 6.00 |
| MERCARE ® | 130180 | (1) | Tocopheryl acetate | 1.00 |
| Tocopherol acetate | | | | 1.00 |
| Dow Corning 1403 fluid | | (5) | Dimethiconol, dimethicone | 3.00 |
| Propyl-4-hydroxybenzoate | 1.07427 | (1) | Propylparaben | 0.05 |
| C | | | | |
| RonaCare ™ Ectoin | | (1) | (Ectoin) | 1.00 |

Manufacture:

All constituents of Phase B were charged together, heated (60-70° C.) and well stirred until a homogeneous mass was obtained. Then Phases B and C were added and stirred again. The homogeneous mixture was filled at 50-60° C.

Procurement Sources:

(1) Merck KGaA

(2) Amoco

(3) Rheox

(4) Cognis GmbH

(5) Dow Corning

### EXAMPLE 17

Cold Sore Cream

| RAW MATERIAL | | INCI | % wt. |
|---|---|---|---|
| RonaCare ™ Ectoin | (1) | (Ectoin) | 1.00 |
| (Art. No. 130200) | | | |
| Aciclovir | | (9-[(2-hydroxyethoxy)- | 5.00 |
| | | methyl]guanin) | |
| Tagat S2 | (2) | PEG-20 glyceryl stearate | 10.00 |
| Lanette O | (3) | Cetearyl alcohol | 20.00 |
| Glycerin (87%) | | Glycerin | 20.00 |
| (Art. No. 104091) | | | |
| Vaseline | (4) | Petrolatum | 35.00 |
| Demin. water | | Aqua (water) | ad 100 |

Manufacture:

All constituents were heated to 75° C. and then cooled to room temperature under stirring.

Procurement Sources:

(1) Merck KGaA

(2) Goldschmidt GmbH

(3) Cognis GmbH

(4) Schumann Sasol

The topical compositions manufactured in Examples 1 to 17 are applied to the skin for the stabilisation of p53 in the skin cells.

1. Use of at least one compound, selected from a compound with the formula 1a, 1b, 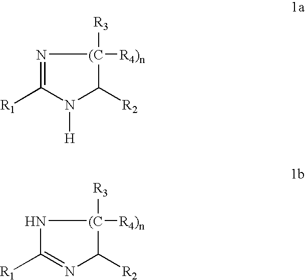
a physiologically compatible salt of it and a stereoisomeric form of it, whereby the following signify
R¹ H or alkyl,
R² H, COOH, COO-alkyl or CO—NH—R⁵,
R³ and R⁴ each mutually independent H or OH,
n 1, 2 or 3,
R⁵ H, alkyl, an amino acid residue, a dipeptide residue or tripeptide residue, and
alkyl an alkyl residue with 1 to 4 carbon atoms,
for the stabilisation of p53.

2. Use according to claim 1 whereby p53 is present as a gene.

3. Use according to claim 1 whereby p53 is present as a protein.

4. Use according to one of the claims 1 to 3 in the form of a topical composition.

5. Use according to one of the claims 1 to 3 in the form of a pharmaceutical composition.

6. Use according to one of the claims 1 to 3 in the form of a nutritional composition.

7. Use according to one of the claims 1 to 4, characterised in that at least one compound used according to claim 1 is present in a topical composition in an amount from 0.0001 to 50% wt. referred to the composition.

8. Use according to one of the claims 1 to 7, characterised in that (S)-1,4,5,6-tetrahydro-2-methyl-4-pyrimidine carboxylic acid and/or (S, S)-1,4,5,6-tetrahydro-5-hydroxy-2-methyl-4-pyrimidine carboxylic acid are used.
